(19) United States
(12) Patent Application Publication
(10) Pub. No.: US 2009/0191272 A1
(43) Pub. Date: Jul. 30, 2009

(54) Use of vegetable fine grain sized fibres for preparing a nutritional composition for reducing mycotoxin bioavailability

(76) Inventors: Emmanuel Kossi Tangni, Louvain-La-Neuve (BE); Yvan Larondelle, Bruxelles (BE); Ludovic De Meeûs d'Argenteuil, Chastre (BE); Nabilla Aoudia, Alger (DZ)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/585,431

(22) PCT Filed: Jan. 6, 2005
(86) PCT No.: PCT/FR2005/000020

§ 371 Date: Sep. 30, 2008

(30) Foreign Application Priority Data
Jan. 7, 2004 (FR) 0400090

(51) Int. Cl. A61K 9/14; A61K 36/00; A61P 3/02

(52) U.S. Cl. 424/489; 424/725

(57) ABSTRACT

The invention relates to using substantially insoluble vegetable fibres embodied in the form of microparticles at least 90% in weight of which has a size less than 700 μm as ingredients for preparing a nutritional composition for reducing a mycotoxin bioavailability in a human being or an animal during ingestion of a food contaminated by said mycotoxins.

The present invention relates to the use of very fine grain sized plant fibers for preparing a nutritional composition for reducing mycotoxin bioavailability.

The importance of the innocuousness of foods for nutritional safety has been widely recognized, in particular by the various governments which, in 1992, participated in the International Conference on Nutrition which took place in Rome (Italy), and in 1996 participated in the World Food Summit in Rome (Italy).

The quality and the safety of foods can be threatened by a large number of factors, including by the presence of natural toxins. Specifically, among the long list of toxins that may naturally be present in common food products, mycotoxins represent a very important category which is among those most widely studied insofar as their ubiquitousness and their harmful effects on human and animal health are the cause of general concern (FAO, 1999, “Preventing Mycotoxin contamination”, publication no. 23, Rome, Italy, p. 55).

Agriculture products are potential targets for pests and diseases. They carry a microbial flora which is variable and numerous, comprising mainly bacteria, yeasts and filamentous fungi. Their presence can in particular cause a deterioration of the quality of the agriculture products, sometimes ranging as far as purely and simply their destruction. Among these microorganisms, certain filamentous fungi are responsible for the production of mycotoxins, which is observed during the growth of agricultural products in the fields or else during their storage under helpful moisture and temperature conditions. The main genera of mycotoxin-producing fungi are Penicillium, Fusarium, Aspergillus and Alternaria.

Mycotoxins are secondary metabolites which have a very variable chemical composition but, in general, have a low molecular weight. Their harmful effects on human health, whether they are acute or chronic, are also very varied. Their targets are mainly the kidneys, the liver, the gastrointestinal tract and the nervous and immune systems. To date, approximately five hundred mycotoxins have been discovered and their number is continually increasing as research advances. However, only about twenty are clearly identified as a real threat to food safety. Among the various families of mycotoxins encountered in food products, mention may in particular be made of aflatoxins (AFLAs), ergot toxins, fumonisins (FBs), ochratoxin A (OTA), patulin (PAT), sterigmatocystine, zearalenone (ZEA) and trichotecenes, including deoxynivalenol (DON). Depending on their nature, these mycotoxins can have harmful and various effects on human or animal health; they can in particular be hepatotoxic and immunotoxic, carcinogenic, teratogenic, neurotoxic or nephrotoxic, or alternatively can lead to digestive conditions or hemorrhages.

The main food products liable to be contaminated with mycotoxins are cereals, nuts, dry fruits, coffee, cocoa, spices, oleaginous seeds, peas and beans, and also fruits. Their derived products can therefore be contaminated, depending on the stability of the toxin during the conversion process. As a result of this, these mycotoxins, and in particular OTA, can be transmitted to numerous products for animal or human consumption, such as animal meals, wine, beer, bread and products derived from coffee and from cocoa (Abarca M L. et al., J. Food Prot., 2001, 64(6), 903-906; Walker R., Av. Exp. Med. Biol., 2002, 504, 249-255). There is also a not insignificant risk of secondary contamination by certain products of animal origin, such as meat and offal from monogastric animals (Pittet A., Rev. Med. Vet., 1998, 149, 479-492).

Mycotoxins are very stable compounds resistant to the majority of agrofood products' conversion processes. Consequently, and given their harmful effects on health, it is of the greatest importance to be able to have effective means for:
either preventing the contamination of food products,
or decontaminating them before and/or after conversion thereof.

The first approach is not always realizable given the growth or storage conditions of the starting materials.

The second approach must therefore be carried out at the industrial level and various physicochemical or biological processes have already been proposed in this respect. These decontamination processes can be classified as three main categories:

1) the first category consists in degrading the toxins into less toxic or nontoxic products so that ingestion thereof is less prejudicial to the organism;
2) the second category consists in subjecting the food to a microfiltration step. By way of example, mention may in particular be made of U.S. Pat. No. 5,248,382, which describes a method for reducing the mycotoxin content in fruit juices, and in particular the patulin content, by means of a filtration on a microporous resin capable of retaining patulin by chemisorption, and in which the pore diameter is less than 20 angstroms. Although effective, this method has the drawback of using a specific and expensive material and of being applicable only to food products in a liquid form;
3) the third category consists in using adsorbents in order to retain, at least partly, the mycotoxins. These adsorbents are either added during the manufacture of the food products and then eliminated therefrom before consumption (generally by filtration) in order to reduce the mycotoxin content in the final food; or added to the consumed foods in order to decrease the mycotoxin bioavailability during digestion.

In the numerous documents of the prior art which describe processes belonging to this third category, the mycotoxins are eliminated through the action of nonbiological, generally inorganic, adsorbents.

Among such inorganic adsorbents, the use of the following has in particular already been proposed:
hydrated sodium calcium aluminosilicates (HSCAS) which are capable of adsorbing aflatoxins present in a medium. However, they are less effective on OTA (Huwig A. et al., Tox. Letters, 2001, 122, 179-188);
phyllosilicates (Diaz D. E. et al., J. Dairy Sci., 1999, 82 (suppl. 1), 838;
active charcoals for which in vivo tests show a preventive effect with respect to aflatoxicoses and an increase in performance levels of the animals when they are fed with contaminated feeds (Galvano et al., J. Food Prot., 1997, 60, 985-991), and also a decrease in the OTA content in the tissues, the blood and the bile of animals subsequent to the incorporation of the adsorbent into diets (Ramos A. J. and Hernandez E., Animal Feed Science and Technology, 1997, 62, 263-269; Huwig et al., 2001, mentioned above);
resins such as cholestyramine and polyvinyl-pyrrolidone, which are also capable of fixing OTA (Piva A. and Galvano F., 1999, “Nutritional approaches to reduce the impact of mycotoxins”, Proceedings of Alltech's 15th Annual Symposium, T. P. Lyons and K. A. Jacques (eds.), 381-400, Nottingham University Press, Nottingham, UK).

On the other hand, the in vivo studies do not reveal a systematic protection by these adsorbents with respect to the toxicity of mycotoxins and in particular of OTA. In fact, the study carried out by Bauer J. (Monatsh Veterinarmed, 1994, 49, 175-181) demonstrated no reduction in the blood and tissue concentration of OTA in pigeons, when the ingestion of foods contaminated with this toxin was combined with the taking of bentonite (German patent application DE 3 810 004), of HSCAS, of cholestyramine or of yeast walls. The same observations were made by Scheideler S. E. (Poultry Science, 1993, 72, 282-288) and Ledoux D. R. et al., 2001 (“In vitro binding mycotoxins by adsorbents does not always translate into in vivo efficacy.”. In: Mycotoxins and phycotoxins in perspective at the turn of the millennium. Proceedings of the X^{th} International IUPAC Symposium in Mycotoxins and Phycotoxins, edited by W. J. de Koe, R. A. Samson, H. P. Van Egmond, J. Gilbert and M. Sabino, Wageningen, The Netherlands, 279-287). More recently, Santin E. et al. (J. Appl. Poultry Res., 2002, 11(1), 22-28) have demonstrated that the addition of HSCAS at a dose of 0.25% scarcely improves the negative impact of OTA (2 ppm) on the physiological parameters in chickens.

Moreover, the ligands mentioned above can bring about losses of nutritive elements and modifications of the organoleptic quality of foods.

Furthermore, it remains that the complete elimination of mycotoxins is impossible. For this reason, it appears to be important to add to the panel of preventive and corrective measures disclosed above, by using agents which are harmless to the organism and organoleptically acceptable, and which are capable of binding the mycotoxins so as to reduce the bioavailability thereof in the organism after ingestion of a contaminated food.

The inventors gave themselves the aim of providing a biological method of detoxification of nutritional media for remedying all of these drawbacks, and which results in a reduction in mycotoxin bioavailability after ingestion of contaminated food products.

On this occasion, the inventors have demonstrated, surprisingly, that the adsorption capacity of essentially insoluble plant fibers, with respect to mycotoxins, is greatly improved when plant fibers in the form of microparticles, at least 90% by weight of which are less than or equal to 700 μm in size, are selected, and said capacity can be taken advantage of in order to prepare a nutritional composition capable of significantly reducing the bioavailability of mycotoxins liable to be present in ingested food products.

A subject of the present invention is therefore the use of essentially insoluble plant fibers in the form of microparticles, at least 90% by weight of which are less than 700 μm in size, as an ingredient in the preparation of a nutritional composition for reducing mycotoxin bioavailability in humans or animals when a food liable to be contaminated with said mycotoxins is ingested.

The inventors have in fact observed that the in vitro adsorption capacity of these plant fibers with respect to mycotoxins is maintained in vivo and makes it possible to reduce the bioavailability and, consequently, the harmful effects of the mycotoxins after ingestion in the organism. The inventors have, moreover, noted, in experiments carried out in vitro, that the use of plant fibers corresponding to these characteristics in terms of grain size makes it possible to significantly increase the adsorption capacity of the plant fibers with respect to the mycotoxins, compared with plant fibers greater than 700 μm in size.

According to the invention, the term “essentially insoluble” is intended to mean a composition in which the soluble fraction of the fibers (determined by enzymatic analysis) does not exceed 55% of the total fiber content. Moreover, in the detailed description which will follow, and unless otherwise explicitly indicated, the terms “plant fibers” refer to essentially insoluble plant fibers having the grain size characteristics as defined above which can be used in the context of the invention.

According to an advantageous embodiment of the present invention, the plant fibers that can be used are preferably in the form of microparticles, approximately 90% by weight of which are less than or equal to approximately 400 μm in size, and even more preferably between approximately 2 μm and 200 μm, inclusive, in size; the grain size being measured by sieving on an A 200 LS Air Jet Sieve sold by the company Alpine (Augsburg, Germany), the fibers being subjected to an air depression of 250 to 700 mm of water column for 6 minutes.

When the plant fibers do not naturally exhibit such a grain size, then they can in particular be prepared by micronization according to the process described, for example, in patent application FR 2 433 910 or according to any other process for obtaining the desired grain size as defined above. The shape of the microparticles is not essential with respect to their adsorption capacity. Thus, the plant fiber microparticles that can be used according to the invention can have a spherical or substantially spherical shape or else can be in the form of more or less long filaments.

When they are derived from a process using a micronization step, then said microparticles have a spherical or substantially spherical shape.

According to an advantageous embodiment of the invention, said nutritional composition is more particularly for reducing the bioavailability of hydrophobic mycotoxins, the inventors having in fact noted, in vitro, that hydrophobic mycotoxins have a greater affinity for the plant fibers than hydrophilic mycotoxins. In this respect, aflatoxins and ochratoxin A are examples of hydrophobic mycotoxins.

According to one embodiment of the invention, the plant fibers are chosen from fibers derived:
from nutritional plants chosen from cereals, leguminous plants, edible plants, fruits, including tropical fruits, and more generally any plant used for nutritional purposes,
from plants used by the paper industry, such as trees, sugarcane, bamboo and cereal straw.

Among the plant fibers derived from cereals, mention may in particular be made of wheat, barley, oats, maize, millet, rice, rye and sorghum fibers, and malted equivalents thereof.

According to the invention, the term “malted equivalent” is intended to mean the germinated grains whose germination has been stopped by means of a thermal treatment and which have then had their germs removed.

Among the fibers derived from nutritional plants other than cereals, mention may in particular be made of fibers derived from apples, pears, grapeseeds, lupin and soya seeds, tomatoes, peas, coffee, etc.

Among these plant fibers, preference is most particularly given to:
the micronized wheat fibers sold under the trade names Realdyme® and Realdyme® M, the latter being in the form of microparticles, at least 90% by weight of which are less than or equal to 100 μm in size;
the micronized oat fibers sold under the trade name Realdyme® A;
the micronized barley fibers sold under the trade name Realdyme® O, and in particular Realdyme® OF and Realdyme® OM;
the micronized apple fibers sold under the name Realdyme® P.

All these fibers are available from the company REALDYME (Garanciéres en Beauce, La Haute Epine, France).

The nature of the fibers used in accordance with the invention is preferably chosen according to the nature of the mycotoxin(s) liable to be present in the nutritional medium, and for which it is desired to reduce the bioavailability after ingestion.

Thus, when the nutritional composition is mainly for reducing the bioavailability of ochratoxin A, aflatoxins, fumonisin and/or deoxynivalenol, then the plant fibers will preferably be chosen from micronized wheat fibers such as the Realdyme® and Realdyme® M products, micronized oat fibers such as, for example, the Realdyme® A product, and mixtures thereof. According to the invention, as regards the reduction in the bioavailability of ochratoxin A, Realdyme® M is most particularly preferably used.

The nutritional composition that can be used according to the invention is intended both for animal nutrition and for human nutrition.

The nutritional composition in accordance with the invention can be in several forms, such as a food supplement, a food ingredient (or intermediate food product: IFP) or a starting material.

When the nutritional composition in accordance with the invention is in the form of a food supplement, then the amount of plant fibers in said supplement can represent up to 100% by weight of the total weight of said supplement; this amount being preferably between 80% and 100% by weight.

When the nutritional composition is in the form of an IFP, it is then preferably intended for human nutrition and is used as an ingredient during the manufacture of a food product liable to be contaminated with mycotoxins. In this case, the nutritional composition then represents from 0.05% to 20% by weight, and even more preferably from 0.1% to 5% by weight, relative to the total weight of the finished food product.

When the nutritional composition is in the form of a starting material, it is preferably intended for animal nutrition. The nutritional composition can then be added to the daily food intake which is given to domestic or breeding animals and which is liable to be contaminated with mycotoxins, before ingestion of said food intake. It can also be used as a starting material or an additive during the manufacture of a complete food for domestic or breeding animals. In the latter two cases, the nutritional composition then preferably represents from 0.05% to 10% by weight, and even more preferably from 0.3% to 2% by weight, relative to the total weight of the food intake or of the complete food.

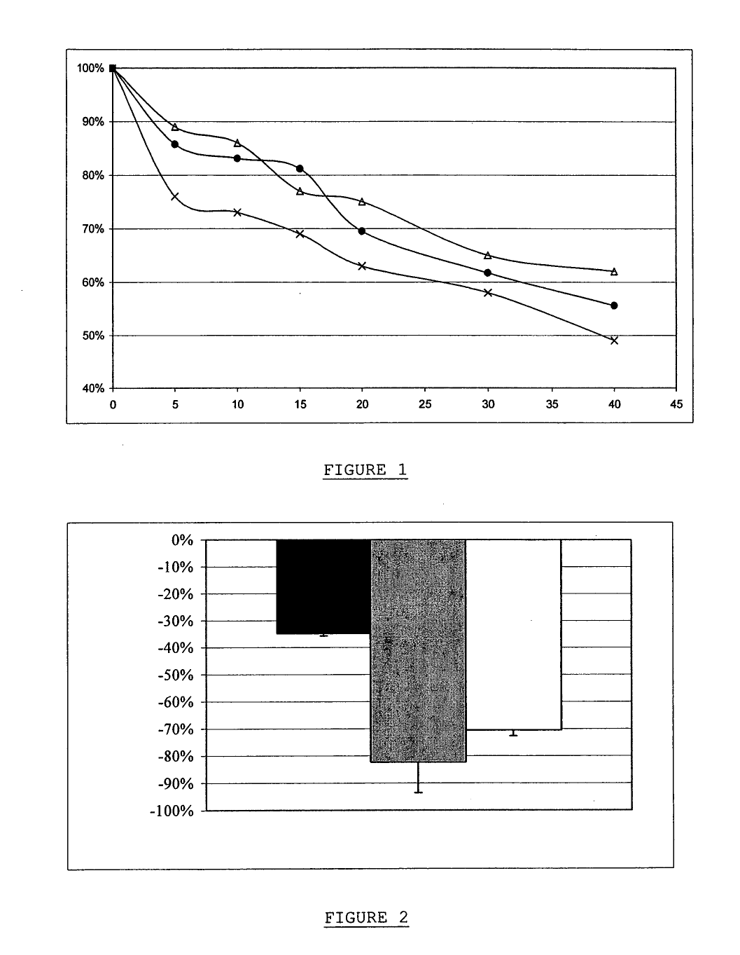

Besides the above arrangements, the invention also comprises other arrangements which will emerge from the description which follows, which refers to an example of demonstration of the OTA adsorption capacity of insoluble plant fibers and preliminary screening of various plant fibers, to an example of a study of the effect of variations in pH on OTA adsorption in a model liquid medium, to an example of demonstration of the adsorption of aflatoxins B1 by insoluble plant fibers in a model liquid medium, to a comparative study of the dose-response effect of the adsorption of AFB1 by micronized and nonmicronized wheat fibers, and to an example of a study of the effect of the incorporation of micronized wheat fibers into a nutritional diet contaminated with OTA, given to rats, on the reduction of OTA bioavailability, and also to the attached FIGS. 1 to 5, in which:

FIG. 1 represents the evolution of OTA adsorption, expressed as residue percentage relative to the amount initially present, by three different fibers (Realdyme® M: cross; Realdyme® OF: solid circles and Realdyme® OM: open triangles) in a model liquid medium containing an initial concentration of 30 ng of OTA/ml for an initial volume of 25 ml and a period of bringing into contact of 45 minutes as a function of the amount of fibers in grams per liter of medium;

FIG. 2 represents the evolution of OTA adsorption (reduction in the amount of OTA as a percentage of the amount initially present) in a model liquid medium, by Realdyme® M fibers, as a function of variations in pH: black bars: pH=6; gray bars: pH=2.2; white bars: after the pH has been increased from 2.2 to 4.8;

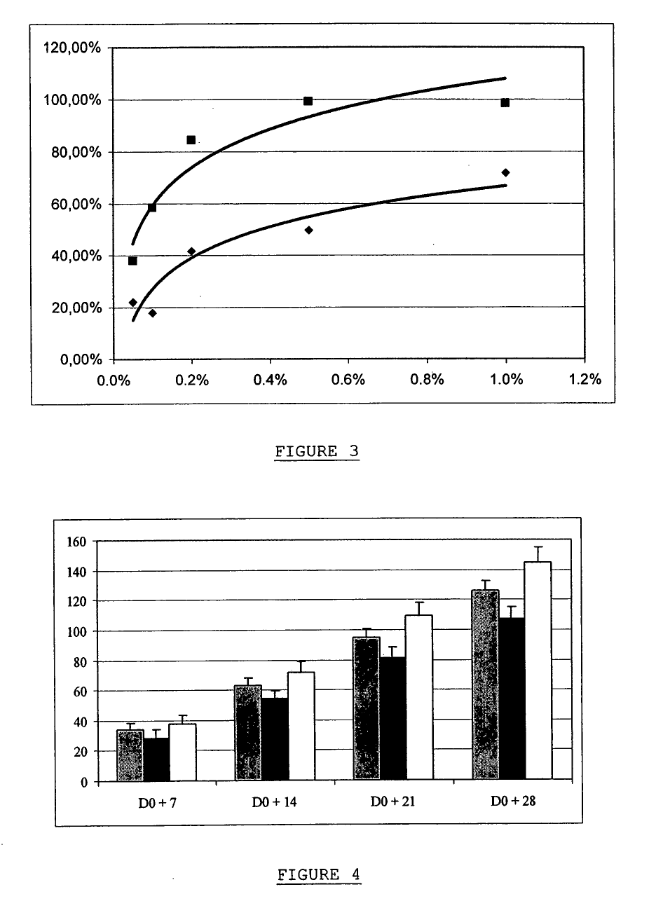

FIG. 3 represents the amount of AFB1 adsorbed in a PBS medium adjusted to pH 3 and initially contaminated with AFB1, this amount being expressed as a percentage of the amount of AFB1 initially present in said medium; black squares: micronized wheat fibers, and black diamonds: nonmicronized wheat fibers;

FIG. 4 represents the cumulative amount of fecal material eliminated (in g) as a function of time (expressed as days after the beginning of the experiment: D0) in rats having received various nutritional diets possibly contaminated with OTA and possibly supplemented with micronized wheat fibers (gray bars: diet 1=nutritional intake not contaminated with OTA; black bars: diet 2=nutritional intake contaminated with OTA, and white bars: diet 3=nutritional intake contaminated with OTA and supplemented with micronized wheat fibers);

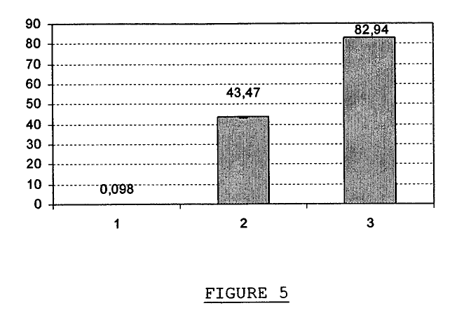

FIG. 5 represents the amount of OTA in the rat feces (in μg) harvested in the fourth week, as a function of the various diets stated above for the description of FIG. 4.

### EXAMPLE 1

### Demonstration of the OTA Adsorption Capacity of Plant Fibers and Preliminary Screening of Various Plant Fibers

Surprisingly, the inventors demonstrated that the incorporation of micronized plant fibers into a model liquid medium makes it possible, through the adsorption of OTA onto the fibers, to reduce the amount of mycotoxins available in this medium. The in vitro tests reported in this example were carried out in order to demonstrate the adsorption capacity of micronized plant fibers when they are incorporated into a liquid medium contaminated with OTA and in order to determine the contact time necessary for optimal adsorption of the OTA by these fibers. Subsequently, a screening of three different plant fibers was carried out in order to determine which fibers were most effective with respect to OTA adsorption. The adsorption capacity of Realdyme® M was, moreover, compared with that of the nonmicronized starting material (nonmicronized wheat fibers).

### 1) Experimental Protocol

A given amount of plant fibers (approximately 20 g/l) is mixed in a sterile 50 ml tube with 25 ml of an aqueous solution containing 2% of dextrose (sold under the trade name D(+) glucose monohydrate by the company Merck), 5% of yeast extract (sold under the trade name Extrait de levures en Poudre [powdered yeast extract] by the company ICN Biomedical) and 1% of peptone (sold under the name Peptone by the company Duchefa), sterilized beforehand at 121° C. for 15 minutes. This aqueous solution has a pH of between 6.0 and 6.2 and is called “DYP” in the subsequent text (model medium). The DYP solution is then contaminated with a variable amount of a solution of OTA in ethanol. The concentration of OTA in the model liquid medium is 57 ng/ml. The content of the tube is then homogenized by manual shaking for 30 seconds and the tube is then placed on a shaker at 90 revolutions per minute (rpm) in a chamber thermostated at 25° C. for 45 minutes. A control treatment without adsorbent (control), i.e. without plant fiber, is carried out for each experiment, and each of these experiments is carried out three times.

The suspension is then centrifuged at 1830 g for 10 minutes at a temperature of 25° C., and the pellet is then separated from the supernatant. 1 ml of supernatant is then extracted, in a sterile tube, with 9 ml of a methanol:water (50:50; v/v) solution. The tube is then vortexed for 30 seconds, and then centrifuged for 10 minutes at 820 g at a temperature of between 5 and 10° C. This extract is then diluted, filtered, and analyzed by high performance liquid chromatography (HPLC).

The HPLC system consists of a Perkin Elmer® LC049 isocratic system pump sold by the company Norwalk (USA) with a 50 μl injection loop sold under the name Rheodyne® by the company Cotati (USA), equipped with a C₁₈ column 150 mm in length and 4 mm in diameter, sold under the name Hypersil® BDS, with a porosity of 3 μm, sold by the company Tracer Analytica (Spain), with an RF 551 fluorescence detector sold by the company Shimadzu (Japan) provided with a xenon lamp having a power of 150 W set at an excitation wavelength (λ_{excitation}) of 332 nm and at an emission wavelength (λₑₘᵢₛₛᵢₒₙ) of 462 nm, and with an SP4290 integrator sold by the company Spectra Physics (USA). The mobile phase is composed of an acetonitrile/water/acetic acid (450/540/10; v/v) mixture filtered through a 0.25 μm membrane and degassed with helium for 15 minutes. The flow rate of the liquid phase is fixed at 1 ml/min at a pressure of between 2900 and 3000 psi.

The total amount of OTA adsorbed is obtained by the difference between the initial amount and the final amount present in the supernatant.

In this example, the following micronized plant fibers were used: Realdyme® M, Realdyme® OF and Realdyme® OM, at various dosages.

### 2) Results

The results obtained are reported in tables I to IV below, and also in the attached FIG. 1.

The percentages of OTA adsorbed onto the Realdyme® M fibers as a function of the period of bringing into contact, with the model medium containing 57 μg/l of OTA, at a pH of between 6.0 and 6.2, are reported in table I below:

TABLE I

| | Amount of mycotoxins adsorbed (%) | | |
|---|---|---|---|
| | Duration of the period of | | |
| | bringing into contact (hours) | | |
| Amount of Realdyme ® M (g/l) | 3 | 24 | 48 |
| 0 (control) | 0 | 0 | 0 |
| 10 | 46.7 | 52.5 | 49.8 |
| 16 | 59.8 | 65.3 | 61.6 |
| 20 | 68.9 | 69.7 | 71.7 |
| 30 | 68.0 | 72.3 | 73.6 |

These results show that the adsorption by the fibers does not vary between 3 and 24 hours. Moreover, the amount of OTA adsorbed increases as a function of the amount of fibers present in the medium.

The effects of periods of bringing into contact of less than 24 hours, on the degree of OTA adsorption (as %) by the fibers (20 g of Realdyme® M fibers per liter of model liquid medium at pH 5.2 containing 35 ng of OTA/ml) are reported in table II below:

TABLE II

| Period of bringing into contact | |
|---|---|
| (in minutes) | % OTA absorbed |
| 0 | 0 |
| 5 | 21 |
| 15 | 20 |
| 45 | 25 |
| 90 | 29 |
| 169 | 28 |
| 360 | 30 |
| 1440 | 43 |

These results show that the adsorption occurs very rapidly (between 5 and 45 minutes, approximately) and that the latter is maintained at least throughout the experiment.

The effects of micronization on the amount of OTA adsorbed by Realdyme® M fibers and of the nonmicronized starting material for said fibers are reported in table III below:

TABLE III

Amount of OTA adsorbed (%)

| | Nonmicronized | |
|---|---|---|
| Amount of fibers (g/l) | starting material | Realdyme ® M |
| 20 | 17% | 33% |
| 30 | 22% | 37% |

These results show that micronization makes it possible to improve the adsorption capacity of the fibers by a factor close to two.

The attached FIG. 1 represents the adsorption capacity of three different fibers (Realdyme® M: cross; Realdyme® OF: solid circles and Realdyme® OM: open triangles) in DYP medium containing an initial concentration of 30 ng of OTA/ml for an initial volume of 25 ml and a period of bringing into contact of 45 minutes. In this figure, the percentage of residual OTA is expressed, for each fiber, as a function of the amount of fibers in grams per liter of the DYP medium.

The results represented in FIG. 1 show that, even at concentrations as low as 5 g of fibers per liter of medium, good adsorption of OTA is observed, in particular with the Realdyme® M fibers.

### EXAMPLE II

### Study of the Effect of pH on the Adsorption of Mycotoxins by Wheat Fibers

The pH of the medium is capable of influencing the adsorption of OTA onto the fibers since it influences the distribution of electrical charges on the fibers, on the toxins and in the medium. During digestion, the pH of the food bolus decreases to a large extent.

In order to study the impact of pH on the adsorption capacity of the fibers, an experiment in a model medium was carried out, consisting in measuring the adsorption before and after a decrease and then a re-increase in pH.

### 1) Experimental Protocol

A known amount of Realdyme® M fibers corresponding to a concentration of 20 g of fibers/l is mixed in a 50 ml sterile tube with 25 ml of DYP model medium as described above in example 1, contaminated beforehand with 50 ng of OTA/ml by means of a solution of OTA in ethanol. The pH of the medium is measured at 6.

The content of the tube is then incubated, separated, extracted, purified and analyzed as described above in example 1.

In parallel, the pH of this same DYP is, in two other tubes already containing fibers, decreased to a value of 2.2 by adding solid lactic acid. The content of one of the two tubes is then incubated, separated, extracted, purified and analyzed as described above in example 1.

The pH of the medium in the second tube is then increased back up to 4.8 by adding sodium hydroxide granules. The content of the tube is then incubated, separated, extracted, purified and analyzed as in example 1.

Each experiment is carried out three times.

### 2) Results:

The results obtained are reported in the attached FIG. 2, which represents the evolution of OTA adsorption (decrease in the amount of OTA in the DYP medium as a % of the amount initially present) onto the fibers after the decrease and after the re-increase in pH in the DYP medium. In this figure, the black bar corresponds to the measurements carried out at pH 6, the grey bar to the measurements carried out at pH 2.2 and the white bar to the measurements carried out after bringing the pH back up from 2.2 to 4.8.

It is clear that the lower the pH, the greater the adsorption of OTA by the fibers, reaching, even for a pH of 2.2, a percentage adsorption of 82.4%. The release of the toxin when the pH is re-increased does not appear to be as great as the increase in adsorption when the pH is decreased.

The decrease in pH therefore makes it possible, for the same amount of fibers, to considerably increase the amount of OTA adsorbed by these fibers.

### EXAMPLE III

### Demonstration of the Adsorption of Aflatoxins B1 by Insoluble Plant Fibers

A given amount of plant fibers (Realdyme®: 20 g/l) is introduced into a sterile 50 ml tube and mixed with 25 ml of phosphate buffer at pH 7 (PBS), contaminated beforehand with aflatoxins B1 (approximately 8.5 ppb). After manual homogenization for 30 seconds, the tube is placed on a shaker at 90 rpm in a chamber thermostated at 25° C. for 45 minutes. A control treatment without adsorbent was used as a control.

At the end of this period, the suspension is centrifuged at 1830 g for 10 minutes at 25° C. and the pellet is then separated from the supernatant. The assay is carried out three times.

The aflatoxins B1 (initial and residual) are analyzed by a direct competition ELISA immunochemical method using the high-sensitivity quantitative specific assay sold under the trade name Veratox® HS by the company Neogen Corporation (USA). The protocol recommended by the supplier of this assay was used.

This immunochemical assay (ELISA) was carried out in the following way:
deposition of 100 μl of conjugate in each microwell not coated with a layer of antibodies;
addition of 100 μl of standard or of 100 μl of sample and mixing;
removal of the entire mixture and deposition thereof in a microwell coated with a layer of antibodies;
incubation for 10 minutes at ambient temperature;
washing five times with deionized water;
deposition of 100 μl of substrate;
incubation for 10 minutes at ambient temperature;
addition of 100 μl of “Red Stop” solution provided with the assay for stopping the substrate-enzyme reaction.

In parallel, the same experiment is carried out at pH 3 in a PBS medium (the pH of which has been adjusted to 3 with a sufficient amount of lactic acid) and also a calibration curve with standards.

The optical density of the colorations is then read at a wavelength of 620 nm using a microplate reader sold under the trade name Labsystem Multiscan MCC/340-RS232C (Labsystems, Finland).

The detection limit and the quantification limit for this analytical method are, respectively, estimated at 3 and 10 ppt, while the percentage recovery is 100%.

The results of this assay are presented in table IV below:

TABLE IV

| Period of | Aflatoxins B1 | Aflatoxins B1 |
|---|---|---|
| bringing into | adsorbed at pH 7 | adsorbed at pH 3 |
| contact (min) | (%) | (%) |
| 0 | 0 | 0 |
| 5 | 66 | 72 |
| 25 | 68 | 69 |
| 45 | 68 | 70 |
| 120 | 67 | 74 |

These results show that the Realdyme® fibers make it possible to adsorb a great amount of aflatoxins B1, from 5 minutes of contact, at neutral pH as at acidic pH.

### EXAMPLE IV

### Study of the Dose-Response Effect During The Adsorption of AFB1 by Micronized or Nonmicronized Wheat Fibers

The aim of this example is to determine the dose starting from which the micronized wheat fibers (REALDYME®) and nonmicronized wheat fibers adsorb the same amount of AFB1.

The experiment was carried out in PBS buffer at pH 3 (the pH being adjusted with lactic acid). The PBS buffer was initially contaminated with a content of approximately 8 ppb of AFB1.

The dose-response effect was evaluated for doses of 0.5%, 1%, 2%, 5% and 10% by weight of each of the two fibers studied; the assaying of AFB1 being carried out as described above in example 3.

The results obtained are represented in the attached FIG. 3, in which the adsorption of AFB1, expressed as percentage reduction in AFB1 concentration in the supernatant relative to the concentration initially present, depends on the amount of fibers used (as % by weight); the black squares correspond to the micronized wheat fibers, the black diamonds correspond to the nonmicronized wheat fibers.

These results show that the micronized wheat fibers are clearly more effective with respect to AFB1 adsorption.

For a commercial point of view, it is advantageous to note that the dose of 0.75% of micronized wheat fibers has the same effect as the dose of 5% of the same fiber which is nonmicronized. These amounts both adsorb 50% of AFB1 of the PBS medium at pH 3 initially contaminated at approximately 8 ppb. Consequently, the use of micronized plant fibers is of great commercial value insofar as this makes it possible to reduce the amount of starting material necessary for the adsorption of a given amount of mycotoxins.

### EXAMPLE V

### Demonstration of the Effect of the Administration of a Nutritional Composition Comprising Micronized Plant Fibers on the Reduction of OTA Bioavailability in Rats

This example was carried out with the aim of showing that the effects observed in vitro, in particular in example 1 above, in terms of adsorption of mycotoxins by plant fibers, are conserved in vivo and make it possible to reduce mycotoxin bioavailability after ingestion of a contaminated food.

More particularly, the present example aims, firstly, to evaluate the effect of these biological adsorbents on growth performance levels and on the amount of fecal material in rats exposed to the toxin via a naturally contaminated nutrition. Secondly, it aims to evaluate the protective effect of these fibers on OTA contents in the blood and the organs.

Specifically, this example aims to:
test the impact of the incorporation of the micronized wheat fibers into the nutrition naturally contaminated with OTA, on the growth and the evolution of the body mass of rats exposed to the toxin;
test the impact of the incorporation of the micronized wheat fibers on the amount of fecal material eliminated in the presence of the OTA;
evaluate the capacity of the micronized wheat fibers to reduce the amount of OTA in the blood plasma, the kidneys and the liver of rats naturally exposed to OTA;
test the impact of the incorporation of the micronized wheat fibers on the OTA content in the fecal material.

### I) Materials and Methods

### 1) Products

potato-extract, dextrose/glucose and agar (agar-agar) (powders) sold by the company Scharlan Chemie S.A., Barcelona, Spain;
powdered peptone (Duchefa, Haarlem, The Netherlands);
ochratoxin A (0-1877) (Sigma Chemical Co., St Louis, Mo., United States);
toluene for analysis (Lab Scan, Dublin, Ireland);
chloroform (analytical grade) (Lab Scan, Dublin, Ireland);
ether (Lab Scan, Dublin, Ireland);
HPLC-grade methanol (Acros Organics, Geel, Belgium);
HPLC-grade acetonitrile (Lab Scan, Dublin, Ireland);
phosphate buffer: phosphate buffered saline-PBS (120 mmol/l NaCl, 2.7 mmol/l KCl, 10 mmol/l phosphate buffer; pH 7.4) (Sigma Chemical Co., St Louis, Mo., United States);
sodium bicarbonate (Merck, Darmstadt, Germany);
sodium chloride (Merck, Darmstadt, Germany);
Milli-Q Plus water (Millipore, Molsheim, France);
99-100% acetic acid for analysis (UCB, Brussels, Belgium);
orthophosphoric acid (analytical grade) (UCB, Brussels, Belgium);
talc (compressed gas) of nitrogen and helium (Air Liquide, Liège, Belgium);
sterile 15 ml and 50 ml polypropylene tubes (Falcon) (Greiner-Labortechnik, Frickenhausen, Germany);
Ochratest® immunoaffinity columns (Vicarn, Watertown, Mass., United States);
filter of Millex®-HV GVIIP 04700 type (0.22 μm) (Millipore, Bedford, Mass., United States);
Acrodisk® 13 mm filter for syringes with 0.45 μm nylon membrane (Pall Gelman Laboratory, Karlstein, Germany).

### 2) Equipment

Vac-Elut® support (Analytichem, Harbour City, Calif., United States);
Ultraturax® T-25 basic (IKA-Werke, Janke und Kunkel GMBH & Co, Staufen, Germany);
rotary evaporator <R> (Büchl, Flawil, Switzerland);
Jouan CR centrifuge (Jouan, Saint Nazaire, France);
OPI-4 spectrophotometer (Shimadzu, Kyoto, Japan);
autoclave (Fedegari Autoclavi, Spa-Albuzzano-Pv, Italy);
high performance liquid chromatography (HPLC) system consisting of:
   isocratic system pump: Perkin Elmer LC049 (Norwalk, Colo., United States), with a 50 μl injection loop (Rheodyne, Cotati, Calif., United States);
   column: C18 of 150 mm×4.0 Hypersil® BDS (reverse phase), 3 μm porosity (Tracer Analytica, Barcelona, Spain);
   RF 551 fluorescence detector (Shimadzu, Kyoto, Japan) with a 150 W xenon lamp set at λexcitation: 332 nm and λemission: 462 nm;
   Spectra Physics SP4290 integrator (San Jose, Calif., United States);
   mobile phase: acetonitrile:water:acetic acid (450:540:10), filtered through a membrane (0.25 μm) and degassed with helium (15 minutes);
   flow rate: 1 ml/minute;
   pressure: 2900-3000 psi.

### 3) Micronized Plant Fibers

The plant fibers tested are the Realdyme® M micronized wheat fibers (Realdyme, Garancières-en-Beauce, France) provided in the form of particles, 90% by weight of which are less than 100 μm in size.

### 4) Production of Ochratoxin A

The rats' nutrition was naturally contaminated with OTA produced in the nutrition biochemistry laboratory in close collaboration with the mycology laboratory of the microbiology unit of the Catholic University of Liege (Belgium).

### 4.1. Microorganism

The OTA was produced by culturing the Penicillium verrucosum strain (code MUCL: CWL 44468 of the fungus library of the University of Louvain la Neuve, Belgium) on wheat grains.

### 4.2. Preparation of the Inoculum

The strain was reinitiated for one week by subculturing on a potato-dextrose-agar (PDA) medium consisting of a broth of potato extract (0.4%), dextrose (2%) and agar (1.5%). The PDA was sterilized beforehand in an autoclave (15 min at 121° C.), poured along a slope and conserved at +4° C. in the refrigerator. The conidia produced on PDA after 10 days of culture were detached aseptically using a sterile loop, and suspended in sterile peptonated (9%) water. Two or three successive 1/10 dilutions were essential for counting the conidia by direct examination under a microscope. A suspension load of 104 conidia/ml was used to inoculate the wheat grains at a dose of 2 ml per 60 g.

### 4.3. Culturing

The culturing of the strain was carried out on soft wheat. To do this, the water content of the wheat grains was adjusted to 24-25% by addition of an appropriate volume of water. The culturing was carried out in 250 ml Erlenmeyer flasks containing 60 g of wheat grains and sterilized in an autoclave (121° C. for 20 min). After the inoculation of 2 ml of conidia suspension under aseptic conditions, the Erlenmeyer flasks were incubated in the dark in a chamber thermostated at 22° C. for 24 days. At the end of this period, the samples were sterilized as previously, frozen, and lyophilized in order to facilitate milling. A total production of 2 kg of wheat contaminated at a rate of 22 μg of OTA/g of wheat was thus obtained.

### 4.4. Composition and Preparation of Diets

The base food for the rats was provided by Carfil (Parvan Service PVBA Carfil quality, Oud-Turnhout, Belgium) in 15 kg bags. It consists mainly of proteins (21%), fats (4.5%), cellulose (4%), ash (7.0%), vitamin A (20 000 IE/IU/kg), vitamin D3 (2000 IE/IU/kg) and vitamin E (40 mg/kg).

Starting from this standard food, the three diets shown in table V below were made up:

TABLE V

| | | | Wheat flour | |
|---|---|---|---|---|
| | Base | Noncontaminated | contaminated | Micronized |
| | food | wheat flour (*) | with 22 μg | wheat |
| Diets | (%) | (%) | of OTA/g (%) | fibers |
| 1 | 88 | 12 | 0 | 0 |
| 2 | 88 | 2 | 10 | 0 |
| 3 | 88 | 0 | 10 | 2 |

(*) The OTA content in the starting wheat flour was determined (OTA content < limit of detection (LD)).

For each of these diets, the various constituents are intimately mixed and pressed in the form of cylindrical granules (diameter=5 mm). A stock of 10.8 kg of foods was thus constituted for each diet. The OTA content was determined on a sample of each diet taken at three different times during the experiment. The 3 sub-samples are mixed and milled.

### 5) Animals and Experimental Plan

Thirty-six nine-week-old male SPF rats of the Wistar/AF race were provided by the Janvier Laboratoire breeding center (Genest-St-Isle, France). Upon reception, the rats were weighed (starting body mass ranging between 248.0 and 294.0 g with an average of 268.6±10.5 g) and distributed into labeled individual cages. They were housed in an air-conditioned animal house at 22° C., subjected to a light-dark (alternating) cycle of 12 hours.

After familiarization with the animal house conditions for 7 to 12 days, the rats were divided randomly into 3 groups (n=12).

### 5.1. Preliminary Assays

The preliminary assays were carried out on a sample of 4 additional 3-month-old rats, and were aimed at controlling the methods for taking samples and analyzing the OTA in the plasma, liver and kidneys (by doping). They were also aimed at determining the percentages of recovery of OTA for the various analytical methods developed in the laboratory.

### 5.2. Experimental Plan

The study was carried out according to a case-control experimental plan: the 36 rats were divided up into 3 groups, each group received one of the 3 diets in table IV above.
treatment 1 (blank): uncontaminated diet 1;
treatment 2 (control): contaminated diet 2;
treatment 3: diet 3 contaminated and including micronized wheat fibers (REALDYME®).

For each of the diets, a systematic intake of 22 g of foods per day and per rat was delivered. This consumption remains just below feeding ad libitum. The amounts of foods corresponding to a 48-hour consumption were given to the rats early in the morning, before 8 o'clock (it should be noted that the rats were not fed on the day of sacrifice). The rats had water ad libitum.

From a hygiene point of view, the litter was renewed systematically twice a week and the eliminated fecal material was separated and weighed.

During the follow-up, the rats were weighed every 3 days until the end of the 28th day. At the end of this period of time, the rats were put to sleep with ether and sacrificed by decapitation (with a guillotine) for the purpose of taking blood, liver and kidney samples. The blood was immediately collected in a preheparinized 15 ml polypropylene tube. After centrifugation at 1830 g for 20 min, the blood plasma (2.5-5 ml) was recovered and stored at −20° C. until the OTA was extracted. The kidneys and the liver were weighed, frozen in liquid nitrogen (freeze-clamping) and immediately conserved at −80° C. The rest of the carcass was conserved at −20° C.

### 6) Determination of the OTA in the Various Biological Matrices

### 6.1 Extraction of the OTA in the Cereals and the Food Granules

The extraction of the OTA from the cereals and the food granules is based on the technique (ISO FDIS1541.2) of the European Committee for Standardization (CEN/TC 275, 1998) of the European Union. The protocol used is as follows:
weigh 50.0 g of meal into a centrifuge tube (cleaned and dried beforehand);
add 200 ml of chloroform and 20 ml of phosphoric acid (0.1 M, pH 3);
homogenize for 3 minutes at 13 500 revolutions per minute (rpm) using an Ultraturax®;
centrifuge for 10 minutes at 820 g at low temperature (5-10° C.);
recover the entire chloroform phase;
perform a second extraction, by repeating the above points;
recover and combine the two chloroform phases;
remove a 350 ml sample of the recovered chloroform phase;
evaporate off the chloroform in a rotary evaporator (30-40° C.);
add 100 ml of sodium carbonate solution (0.5 M, pH=9) to the evaporation residue;
centrifuge for 10 minutes at 820 g at low temperature (5-10° C.);
recover 20 ml of this bicarbonate solution and purify it on an immunoaffinity column in accordance with the protocol described in point 6.4 below.
   6.2. Extraction of the OTA from the Blood Plasma
take a 2.5 ml sample of plasma;
add 20 ml of chloroform and 10 ml of phosphoric acid (0.1 M, pH 3);
homogenize for 3 minutes using a vortex;
centrifuge for 10 minutes at 820 g at low temperature (5-10° C.);
recover the entire chloroform phase using a pipette;
perform a second extraction, by repeating the above points;
recover the entire chloroform phase using a pipette and combine the two chloroform phases;
evaporate off the chloroform using a rotary evaporator (30-40° C.);
add 20 ml of sodium carbonate solution (0.5 M, pH=9) to the evaporation residue;
centrifuge for 10 minutes at 820 g at low temperature (5-10° C.);
recover a volume of 15-20 ml of this bicarbonate solution and purify it on an immunoaffinity column according to the protocol described below in point 6.4.
   6.3. Extraction of the OTA from the Kidneys, the Liver and the Feces

In the following text and unless otherwise indicated, the instructions below are common to the kidneys, the liver and the feces.
weigh into a centrifuge tube:
   for the kidneys: 2.5 g of kidneys,
   for the liver: the entire organ,
   for the feces: 4 g of matter;
for the kidneys and the liver only: add 2 g of sodium chloride;
add 50 ml of chloroform and 20 ml of phosphoric acid (0.1 M, pH 3);
homogenize for 3 minutes at 13 500 rpm using an Ultraturax®;
centrifuge for 10 minutes at 820 g at low temperature (5-10° C.);
recover the lower organic phase;
perform a second extraction, by repeating the above points;
evaporate off the chloroform using a rotary evaporator, at a temperature of between 30 and 40° C.;
for the kidneys and the feces only: add 100 ml of sodium carbonate solution (0.5 M, pH=9) to the evaporation residue;
for the liver only: add 50 ml of sodium carbonate solution (0.5 M, pH 9) to the evaporation residue;
centrifuge for 10 minutes at 820 g at low temperature (5-10° C.);
recover a volume of 15-20 ml of this bicarbonate solution and purify it on an immunoaffinity column as described below in point 6.4.
   6.4. Purification on an immunoaffinity column (Ochra Test®)
fix the column onto a Vac-Elut® support and surmount it with a 20 ml syringe by means of an adaptor;
condition the immunoaffinity column with 20 ml of PBS solution;
pass 15-20 ml of the bicarbonate solutions over the column at a flow rate of 1 to 2 ml/minute;
wash the column with 20 ml of Milli-Q plus water;
recover the OTA by eluting with 2 ml of methanol and 2 ml of Milli-Q plus water;
pass 20 ml of air through the column, using a syringe, for the purpose of recovering the entire eluate.

### 6.5. Detection and Quantification by HPLC

One hundred μl of filtered extract were injected in order to completely fill the injection loop, the volume of which is 50 μl. The elution phase was composed of an acetonitrile/water/acetic acid (540/450/10; v/v/v) mixture. The detection of the OTA was carried out at 332 nm (excitation) and at 462 nm (emission). The OTA concentration was determined by interpolation of the peak heights on the calibration line at the same retention time.

In parallel, a calibration line was produced with a diluted stock solution of standards of 0.5, 1, 2, 3, 4 and 5 ng OTA/ml in the water/methanol mixture (50/50; v/v). The calibration line was determined by the least squares method (not represented).

### 7) Analytical Method Performance Characteristics

### Detection Limits

Taking into account all the analytical procedures (extraction, purification and quantification), the detection limits are estimated at 10 ng/kg for the cereals, 20 ng/l for the plasma and 20 ng/kg for the kidneys, the liver and the feces.

### 8) Data Processing and Statistical Analysis

The growth rate of the rats was calculated by virtue of the difference between two consecutive body masses, related to the amount of time separating the two weighings.

The effect induced by the contaminated diets was evaluated by the difference in the results obtained compared with diet 1 (blank).

The nonparametric analytical procedure was used given the heterogeneity of the data obtained. The Kruskal Wallis test made it possible to perform the analysis of variance of the various treatments. The Wilcoxon test was used to assess the difference between the means of the body masses of the rats exposed to the contaminated diets and those of the rats subjected to the blank diet, at the beginning and during the 4 weeks of experimentation, while the Mann-Whitney test was used for the purpose of assessing the difference between the mean of a treatment and that of the blank or of the control. The Spearman correlation test was used to verify the consistency of the relationship between the plasma and kidney OTA contents.

The results are expressed in the form of mean±standard deviation. The data were analyzed by means of the SPSS statistical software version 10.0 (1999). The differences are considered to be significant at p<0.05.

### II) Results

### 1) Food Consumption and Exposure of the Rats to OTA

The results of assaying the OTA in the three diets are given in table VI below:

TABLE VI

| | | OTA | | | |
|---|---|---|---|---|---|
| | | content in | Total OTA | Daily intake of OTA | |
| | | the diet | ingested* | (μg/kg of bm/day) | |
| Diets | Treatments | (μg/kg) | (μg) | Mean ± SD | Range |
| Diet 1 | Blank | 10.3 | 6.345 | 1.99 ± 0.40 | 2.57-1.23 |
| Diet 2 | Control | 2240.67 | 1380.252 | 438.42 ± 84.27 | 559.33-276.51 |
| Diet 3 | Wheat fibers | 2201.64 | 1356.210 | 433.52 ± 84.41 | 555.46-273.23 |

*Exposure time = 28 days; daily consumption of 22 g

These results show that the base food used (Carfil) is slightly contaminated with OTA (10.3 μg/kg) and that the incorporations of naturally contaminated meal into the two other intakes made it possible to obtain a mean contamination of 2221.16 μg of OTA/kg. Already, the base food given at a rate of 22 g per day and per rat provides a daily intake of 1.99±0.4 μg/kg of body mass (bm). The other two food intakes given in the same amount expose the rats to mean daily intakes of 433.52 and 438.42 μg of OTA/kg of bm according to the diet.

2) Impact of the Incorporation of the Micronized Wheat Fibers into the Diets, on the Amount of Fecal Material Eliminated by the Rats Exposed to the Various Diets

The results obtained are reported in the attached FIG. 4. FIG. 4 represents the cumulative amount of fecal material eliminated (in g) as a function of time for each of the diets (gray bars: diet 1; black bars: diet 2 and white bars: diet 3).

These results reveal a significant difference in the amounts of fecal material excreted by the rats subjected to the 3 diets. The multiple comparison test makes it possible to distinguish three classes ordered hereinafter: rats subjected to diet 2<rats subjected to diet 1<rats subjected to diet 3.

The repetitive administration of OTA via diet 2 for 28 days, i.e. without micronized wheat fibers, continuously decreases the amount of fecal material excreted (FIG. 5). On the other hand, the addition of the fibers to the diet increases the excretion of fecal material. In fact, these fibers provide an increase of the order of 35% compared with diet 2 and of the order of 15% compared with diet 1. The increase in the mass of feces is one of the well-known effects of dietary fiber.

### 3) Impact of the Incorporation of Micronized Wheat Fibers on the Concentrations of OTA in the Blood Plasma, the Kidneys, the Liver and the Feces of the Rats Exposed to the Various Diets

### 3.1. Assaying of the Plasma OTA

Table VII recapitulates hereinafter the crude contents of OTA in the plasma, and also those related to the total intakes of OTA during the period of exposure of the rats to the naturally contaminated diets.

TABLE VII

| | | | OTA content in the blood | |
|---|---|---|---|---|
| | OTA content in the blood | | plasma related to the total | |
| | plasma | | amount of OTA ingested | |
| | | % | ng/ml/μg of | % |
| Diets | ng/ml | decrease^{b} | OTA ingested | decrease |
| 1 | 21.1 ± 39.0 | — | 3.3 ± 0.36 | — |
| 2 | 830.3 ± 411.9 | — | 0.60 ± 0.30 | — |
| 3 | 494.1 ± 186.3^{a} | 40.5 | 0.37 ± 0.14 | 49.13 |

^{a}A significant difference is observed between this mean and that of the rats subjected to the control diet 2 (p < 0.05).
^{b}Percentage of the amount of plasma OTA of the rats subjected to diet 3 related to that of the control diet 2.

These results show that the micronized wheat fibers exercise a significant activity on the decrease in the amount of OTA in the blood plasma. It should be noted that the incorporation of the micronized wheat fibers, at a dose of 2%, into the food makes it possible to decrease by 40.5% the OTA concentration in the plasma. This ability is also noted when the contents are related to the total amounts of OTA ingested during the entire period of the experiment.

Consequently, the incorporation of micronized wheat fibers into a food product makes it possible to significantly reduce the OTA bioavailability.

### 3.2. Assaying of the OTA in the Kidneys

The crude OTA contents in the kidneys and those related to the total OTA intakes during the period of exposure of the rats to the naturally contaminated diets are given in table VIII below:

TABLE VIII

| | | | OTA content in the kidneys | |
|---|---|---|---|---|
| | OTA content in the | | related to the total amount | |
| | kidneys | | of OTA ingested | |
| | | % | ng/g/μg of | % |
| Diets | ng/g | decrease^{b} | OTA ingested | decrease |
| 1 | 1.39 ± 1.66 | — | 0.2 ± 0.259 | — |
| 2 | 79.38 ± 31.4 | — | 0.057 ± 0.023 | — |
| 3 | 57.07 ± 42.5^{a} | 28.11 | 0.043 ± 0.032 | 24.56 |

^{a}A significant difference is observed between this mean and that of the rats subjected to the control diet 2 (p < 0.05).
^{b}Percentage of the amount of OTA in the kidneys of the rats subjected to diet 3 related to that of the control diet 2.

These results show that the wheat fibers also exercise a significant activity on the decrease in the concentration of OTA in the kidneys. It should in fact be noted that the incorporation of micronized wheat fibers, at a dose of 2%, into the food makes it possible to decrease by 28.11% the OTA concentration in the kidneys.

The OTA content in the kidneys exhibits a linear relationship with the plasma OTA concentration, illustrated by the following regression equation: OTA_{kidneys}=(15.45±6.512)+(0.081±0.02)*OTAₚₗₐₛₘₐ (R²=0.620; p<0.0001).

If the kidney is the OTA accumulation organ, the contents which are found therein are good indicators of the contamination to which the organism is really subjected over a relatively long period.

### 3.3 Assaying of the OTA in the Liver

The crude OTA contents in the liver and those related to the total OTA intakes during the period of exposure of the rats to the naturally contaminated diets are given in table IX below:

TABLE IX

| | | | OTA content in the liver | |
|---|---|---|---|---|
| | | | related to the total amount | |
| | OTA content in the liver | | of OTA ingested | |
| | | % | ng/g/μg of | % |
| Diets | ng/g | decrease^{b} | OTA ingested | decrease^{b} |
| 1 | 1.43 ± 3.5 | — | 0.24 ± 0.56 | — |
| 2 | 73.68 ± 31.43 | — | 0.0483 ± 0.0515 | — |
| 3 | 45.13 ± 17.38^{a} | 38.58 | 0.0250 ± 0.0452^{a} | 40.05 |

^{a}A significant difference is observed between this mean and that of the rats subjected to the control diet 2 (p < 0.05).
^{b}Percentage of the concentration of OTA in the liver of the rats subjected to diet 3 related to that of the control diet 2.

These results show that the wheat fibers exercise a significant activity on the decrease in the concentration of OTA in the liver. The incorporation of micronized wheat fibers, at a dose of 2%, into the food makes it possible to decrease by 38.58% the OTA concentration in the liver.

### 3.4. Assaying of the OTA in the Feces

Depending on the efficacy of gastrointestinal absorption and on the metabolism in the liver, mycotoxins and their metabolites are preferentially excreted in the urine and feces. The amounts of OTA found in the feces are given in table X below:

TABLE X

| | Amount of OTA in the feces | | Amount of OTA in the feces | |
|---|---|---|---|---|
| | harvested during week 1 | | harvested during week 4 | |
| | | % | | % |
| Diets | μg | increase^{b} | μg | increase^{b} |
| 1 | 0.046 ± 0.0004 | — | 0.098 ± 0.0016 | — |
| 2 | 35.27 ± 0.12 | — | 43.47 ± 0.17 | — |
| 3 | 58.41 ± 0.17^{a} | 65.60 | 82.94 ± 0.13^{a} | 90.80 |

^{a}A significant difference is observed between this mean and that of the rats subjected to the control diet 2 (p < 0.05).
^{b}Percentage of the amount of OTA in the fecal material of the rats subjected to diet 3 related to that of the control diet 2.

The assaying of OTA in the fecal material demonstrates the positive effect of the dietary fibers in the adsorption of OTA. In fact, according to the results obtained (table X), the addition of the biological fibers to the food intake made it possible to increase the amount of OTA eliminated. This increase is 65.60% during the first week and 90.80% for the fourth week.

The crude OTA contents in the fecal material of experimental weeks 1 and 4 and those related to the total OTA intakes during the corresponding periods of exposure of the rats to the naturally contaminated diets are given in tables XI and XII below. The values obtained show once again that the micronized wheat fibers exercise an adsorption activity with respect to OTA.

TABLE XI

| | OTA content in the feces | | OTA content in the feces | |
|---|---|---|---|---|
| | (week 1) | | (week 4) | |
| | | % | | % |
| Diets | μg/g | increase^{b} | μg/g | increase^{b} |
| 1 | 0.0014 ± 0.0004 | — | 0.003 ± 0.0016 | — |
| 2 | 1.251 ± 0.11 | — | 1.68 ± 0.168 | — |
| 3 | 1.75 ± 0.17^{a} | 40.20 | 2.55 ± 0.13^{a} | 51.9 |

^{a}A significant difference is observed between this mean and that of the rats subjected to the control diet 2 (p < 0.05).
^{b}Percentage of the concentration of OTA in the feces of the rats subjected to diet 3 related to that of the control diet 2.

TABLE XII

| | OTA content in the feces | | OTA content in the feces | |
|---|---|---|---|---|
| | related to the total | | related to the total | |
| | amount of OTA ingested | | amount of OTA ingested | |
| | (week 1) | | (week 4) | |
| | ng/g/μg of OTA | % | ng/g/μg of OTA | % |
| Diets | ingested | increase^{b} | ingested | increase^{b} |
| 1 | 0.86 ± 0.08 | — | 2.01 ± 0.34 | — |
| 2 | 3.62 ± 0.65 | — | 4.85 ± 0.14 | — |
| 3 | 5.15 ± 0.91^{a} | 42.27 | 7.48 ± 0.13^{a} | 54.23 |

^{a}A significant difference is observed between this mean and that of the rats subjected to the control diet 2 (p < 0.05).
^{b}Percentage of the concentration of OTA in the feces of the rats subjected to diet 3 related to that of the control diet 2.

The experimental results obtained therefore particularly reflect the effect of the micronized plant fibers on the reduction of OTA bioavailability after ingestion of a contaminated food.

All these results show, consequently, that the micronized plant fibers adsorb and retain mycotoxins not only in model liquid media, but also in the gastrointestinal chyme of animals. The mycotoxin bioavailability is thus reduced.

These fibers are natural elements originating from cereals, which is an advantage for their use in animal or human nutrition.

1. A nutritional composition for reducing mycotoxin bioavailability in humans or animals when a food liable to be contaminated with said mycotoxins is ingested comprising essentially insoluble micronized plant fibers in the form of microparticles, at least 90% by weight of which are less than 700 μm in size.

2. The composition as claimed in claim 1, wherein the micronized plant fibers are in the form of microparticles, at least 90% by weight of which are less than or equal to 400 μm in size.

3. The composition as claimed in claim 2, wherein the micronized plant fibers are in the form of microparticles, at least 90% by weight of which are between 2 μm and 200 μm, inclusive, in size.

4. The use composition as claimed in claim 1 wherein said nutritional composition is for reducing the bioavailability of hydrophobic mycotoxins.

5. The composition as claimed in claim 1 wherein the plant fibers are chosen from fibers derived:
from nutritional plants chosen from cereals, leguminous plants, edible plants and fruits,
from plants used by the paper industry, chosen from trees, sugarcane, bamboo and cereal straw.

6. The composition as claimed in claim 5, wherein the plant fibers derived from cereals are chosen from wheat, barley, oat, maize, millet, rice, rye and sorghum fibers, and malted equivalents thereof.

7. The composition as claimed in claim 5, wherein the fibers derived from nutritional plants, other than cereals, are chosen from fibers derived from apples, pears, grapeseeds, lupin and soya seeds, tomatoes, peas and coffee.

8. The composition as claimed in claim 1 wherein the nutritional composition is for reducing the bioavailability of ochratoxin A, aflatoxins, fumonisin and/or deoxynivalenol, and the micronized plant fibers are chosen from wheat fibers and oat fibers, and mixtures thereof.

9. The composition as claimed in claim 8, wherein the nutritional composition is for reducing ochratoxin A bioavailability, and the plant fibers are micronized wheat fibers in the form of microparticles, 90% by weight of which are less than or equal to 100 μm in size.

10. The composition as claimed in claim 1, wherein the nutritional composition is in the form of a food supplement, and the amount of plant fibers in said supplement represents up to 100% by weight of the total weight of said supplement.

11. The composition as claimed in claim 10, wherein the amount of plant fibers in said supplement is between 80% and 100% by weight of the total weight of said supplement.

12. The composition as claimed in claim 1, wherein the nutritional composition is intended for human nutrition, and that it is in the form of a nutritional ingredient to be added during the manufacture of a food product at a rate of from 0.05% to 20% by weight relative to the total weight of said food product.

13. The composition as claimed in claim 1, wherein the nutritional composition is intended for animal nutrition, and it is in the form of a starting material to be added to the daily food intake which is given to domestic or breeding animals, or to be incorporated, as an ingredient, during the manufacture of a complete food for domestic or breeding animals at a rate of from 0.05% to 10% by weight relative to the total weight of the food intake or of the complete food.

14. A method of preparing a nutritional composition for reducing mycotoxin bioavailability in humans or animals comprising incorporating into a food product essentially insoluble plant fibers in the form of microparticles, at least 90% by weight of which are less than 700 μm in size.

15. The method as claimed in claim 14, wherein the plant fibers are in the form of microparticles, at least 90% by weight of which are less than or equal to 400 μm in size.

16. The method as claimed in claim 14 for reducing the bioavailability of ochratoxin A, aflatoxins, fumonisin and/or deoxynivalenol, wherein the micronized plant fibers are chosen from wheat fibers and oat fibers, and mixtures thereof.

17. The method as claimed in claim 16, wherein the plant fibers are micronized wheat fibers in the form of microparticles, 90% by weight of which are less than or equal to 100 μm in size.

18. The method as claimed in claim 14, wherein the food product is in the form of a food supplement, and the amount of plant fibers in said supplement represents up to 100% by weight of the total weight of said supplement.

19. The method as claimed claim 14, wherein the food product is intended for human nutrition, and the method comprises adding the plant fibers during the manufacture of the food product at a rate of from 0.05% to 20% by weight relative to the total weight of said food product.

20. The method as claimed in claim 14, wherein the food product is intended for animal nutrition, and the method comprises adding the plant fibers during the manufacture of a complete food for domestic or breeding animals at a rate of from 0.05% to 10% by weight relative to the total weight of the food intake or of the complete food.
